(19) United States
(12) Patent Application Publication
(10) Pub. No.: US 2026/0158252 A1
(43) Pub. Date: Jun. 11, 2026

(54) Methods for Administration of Drug to the Retina

(71) Applicant: Georgia Tech Research Corporation, Atlanta, GA (US)

(72) Inventors: Mark R. Prausnitz, Atlanta, GA (US); Seyed Amirhossein Hejri Bidgoli, Atlanta, GA (US)

(21) Appl. No.: 18/706,853

(22) PCT Filed: Nov. 8, 2022
(86) PCT No.: PCT/US2022/049319

§ 371 Date: May 2, 2024

Related U.S. Application Data
(60) Provisional application No. 63/276,966, filed on Nov. 8, 2021.

(51) Int. Cl. A61M 37/00; A61B 3/10

(52) U.S. Cl. CPC A61M 37/0015; A61B 3/10; A61M 2037/0023; A61M 2037/003; A61M 2037/0061; A61M 2202/0007; A61M 2202/04; A61M 2210/0612

(57) ABSTRACT

Methods are provided for administering a therapeutic agent to an eye of a patient, wherein the method includes (i) inserting a microneedle into the eye of the patient, wherein the microneedle extends through the sclera and choroid layers, but not into the vitreous, and without a vitrectomy or a retinotomy; and (ii) injecting a fluid comprising a therapeutic agent through a lumen of the microneedle and into a subretinal space of the eye, wherein the microneedle has (i) a beveled tip with a bevel angle from 40° to 70° and (ii) an outer diameter that is less than 150 μm.

### CROSS-REFERENCE TO RELATED APPLICATIONS

This application claims priority to U.S. Provisional Patent Application No. 63/276,966, filed on Nov. 8, 2021, which is incorporated herein by reference.

### BACKGROUND

Conventional methods for treating degenerative eye disease, particularly those requiring drug delivery to the retina, include intravitreal and subretinal space injections and systemic and topical delivery. Generally, systemic delivery via the blood stream is the most common drug delivery method; however, in the context of ocular delivery, it is ineffective given the limited bioavailability of drugs in the eye from drugs administered systemically. Similarly, eye drops are commonly used in treating anterior segment disorders, but are limited by low bioavailability in the retina and choroid, making topical treatments ineffective for posterior segment diseases.

Thus, more invasive treatments like intravitreal and subretinal space injections are the most common means of drug delivery to the retina. Intravitreal injection is the most common method of delivery to posterior ocular tissues (e.g., the retina), and is performed in an outpatient setting with topical anesthetics. While intravitreal injection is effective in providing higher drug bioavailability in the retina and choroid compared to topical or systemic administrations, there are several problems associated with intravitreal injections. For example, certain diseases or disorders may require frequent injections, as often as once a month, which increases the risk of noncompliance with the desired treatment plan by the patient. There are also complications that may arise from repeated intravitreal injections, such as endophthalmitis (i.e., eye infection), cataract, retinal detachment, intraocular hemorrhage, elevated intraocular pressure, and uveitis. Moreover, only a fraction of the drugs delivered via intravetreal injection may actually reach the retina, ultimately reducing the bioavailability in the targeted tissue.

Subretinal space injection, conversely, provides increased bioavailability in the retina by bypassing drug barriers in the front of the eye; however, conventional means for subretinal space injection suffer from significant problems. Most significantly, surgeons have limited ability to control the penetration depth of the needle tip into the retina, which may cause patient complications. For example, if the needle does not fully penetrate the retina, the drug will ultimately be injected into the vitreous and not the retina. Additionally, if the needle tip penetrates too deeply, there is a significant risk of choroidal hemorrhage, which can lead to long-term complications for the patient. The injection fluid may also separate the retina from the underlying tissue, resulting in the expansion of the subretinal space and formation of a fluid blister commonly referred to as a subretinal bleb. Not only do subretinal blebs increase the risk of retinal detachment, the injection fluid remains concentrated in one space within the retina, decreasing the treatment efficacy.

It therefore would be desirable to provide improved methods of drug delivery to the retina, particularly those that improve upon the safety, practicality, and efficacy of conventional treatments.

### BRIEF SUMMARY

In one aspect, methods are provided for administering a therapeutic agent to an eye of a patient, wherein the method includes (i) inserting a microneedle into the eye of the patient, wherein the microneedle extends through the sclera and choroid layers, but not into the vitreous, and without a vitrectomy or a retinotomy; and (ii) injecting a fluid comprising a therapeutic agent through a lumen of the microneedle and into a subretinal space (SRS) of the eye, wherein the microneedle has (i) a beveled tip with a bevel angle from 40° to 70° and (ii) an outer diameter that is less than 150 μm. In particular embodiments, the method further includes imaging tissue of the eye to identify one or more target sites which have a needle path in which no large conjunctival, choroidal and retinal blood vessels exist; and selecting one of the one or more targets sites as the site for inserting the microneedle. In particular embodiments, the method further includes stabilizing the eye while inserting the microneedle. In some particular embodiments, the injecting is done in manner that suppresses subretinal bleb growth and guides the fluid away from the site of the insertion, and such as toward the posterior retina and/or macula.

In some preferred embodiments, the bevel angle may be from 50° to 60° and the outer diameter from 75 μm to 125 μm; the microneedle may be inserted such that a tip opening of the microneedle is located at the interface of the retina and retinal pigment epithelium (RPE) layers without penetrating deeper than the outer nuclear layer of the retina; and/or the method further includes selecting a length of the microneedle such that the beveled tip of the microneedle is configured to be positioned within the SRS upon complete insertion of the microneedle.

In another aspect, an injection apparatus is provided for administering a therapeutic agent into a subretinal space (SRS) of an eye of a patient, the apparatus including a microneedle which extends from a needle hub and is configured to be inserted through the sclera and choroid layers, but not into the vitreous; and an injector configured to inject a fluid comprising a therapeutic agent through a lumen of the microneedle and into the SRS following insertion of the microneedle; wherein the microneedle has (i) a beveled tip with a bevel angle from 40° to 70° and (ii) an outer diameter that is less than 150 μm. In some embodiments, the needle hub as a width (W) and the microneedle as a length (L), the ratio of W:L is between 0.1 and 10, such as between 0.2 and 5, between 0.5 and 3, between 0.7 and 2, between 0.8 and 1.5, or about 1. The injector may include a reservoir for the fluid and means for driving the fluid from the reservoir into and through the microneedle. The injection apparatus may further include means for stabilizing a position of the microneedle relative to a target site in an eye of a patient; and/or an imaging system configured to image a target site for a microneedle insertion path in which no large conjunctival, choroidal and retinal blood vessels exist.

### BRIEF DESCRIPTION OF THE DRAWINGS

The detailed description is set forth with reference to the accompanying drawings. The use of the same reference numerals may indicate similar or identical items. Various embodiments may utilize elements and/or components other than those illustrated in the drawings, and some elements and/or components may not be present in various embodiments. Elements and/or components are not necessarily drawn to scale.

FIG. 1 depicts a method of subretinal injection, according to the prior art.

FIG. 2A depicts a method of trans-scleral subretinal injection, according to an exemplary embodiment.

FIG. 2B depicts the method of trans-scleral subretinal injection of FIG. 2A, according to an exemplary embodiment.

FIG. 3A is one side view of a microneedle, according to an exemplary embodiment.

FIG. 3B is another side view of the microneedle of FIG. 3A, rotated 90 degrees to view the opening of a hollow bore in the microneedle.

FIG. 4 is a plan view of an injection apparatus, according to an exemplary embodiment.

FIG. 5A is a partial cross-sectional view of a tip portion of an injection apparatus, according to an exemplary embodiment.

FIG. 5B is a partial cross-sectional view of the injection apparatus of FIG. 5A and an imaging system, according to an exemplary embodiment.

FIG. 5C is a partial cross-sectional view of the injection apparatus of FIG. 5A and a microneedle stabilizer, according to an exemplary embodiment.

FIG. 5D is a cross-sectional view of the injection apparatus of FIG. 5A and an eye stabilizer, according to an exemplary embodiment.

FIG. 6A is a cross-sectional view depicting a subretinal bleb resulting from a conventional (prior art) subretinal injection.

FIG. 6B is a cross-sectional view depicting a smaller subretinal bleb resulting from trans-scleral subretinal injection, according to embodiments of the present disclosure.

FIG. 6C is a cross-sectional view depicting fluid in the subretinal space resulting from trans-scleral subretinal injection, according to embodiments of the present disclosure.

FIG. 7 is microphotograph depicting bifurcating dispersion of a fluid injected via trans-scleral subretinal injection, according to embodiments of the present disclosure.

### DETAILED DESCRIPTION

Methods have been developed for administering a therapeutic agent to an eye of a patient, in particular to a subretinal space (SRS) of the eye. The method includes inserting a microneedle into the eye of the patient, wherein the microneedle extends through the sclera and choroid layers, but not into the vitreous, and without a vitrectomy (i.e., removal of most or all of the vitreous from the eye) or a retinotomy (i.e., an incision that traverses most of all of the retina); and injecting a fluid comprising a therapeutic agent through a lumen of the microneedle and into a subretinal space (SRS) of the eye, wherein the microneedle has (i) a beveled tip with a bevel angle from 40° to 70° and (ii) an outer diameter that is less than 150 μm.

It was discovered that flowrate of SRS injected fluid can impact bleb formation and that the injecting can be done in manner that suppresses subretinal bleb growth (as compared, for example, to a conventional injection method) which advantageously may guide the fluid away from the site of the insertion and toward the posterior retina and/or macula. In some embodiments, for example, only 25% to 75% by volume of the injected fluid may form a subretinal bleb, with the rest spread within the SRS, e.g., through the formation of intricate tree-like bifurcating patterns that spread circumferentially from the site of the injection. For example, the fluid may be injected at a flowrate from 0.5 μL/s to 20 μL/s, such as from 2 μL/s to 10 μL/s.

Degenerative eye diseases impact a signification portion of the human population, many of which are only treatable via drug delivery to the retina. The standard method for delivering drugs to the retina or subretinal space typically involve injections via a hypodermic needle, measuring several centimeters in length, which traverse the eye across the vitreous humor and penetrating into the subretinal space across the retina. This method of injection is “trans-vitreal” because the hypodermic needle traverses the vitreous to access the subretinal space. As used herein, “subretinal space” refers to the area between the retinal pigment epithelium and the retina. The term “retinal pigment epithelium” (RPE) refers to the monolayer of pigmented cells between the choroid and the subretinal space.

While this method is effective, the procedure may be quite uncomfortable, requires a costly surgery performed by a highly trained retinal surgeon, and presents serious risks to the patient. However, microneedles adapted for precise subretinal injection may be effective for similarly treating degenerative eye diseases in a manner that is equally as effective as conventional subretinal injections, without the same level of patient risk.

It has been discovered that beveled microneedles having a particular length, width, and tip angle may be effective to achieve trans-scleral subretinal injection without significant tissue damage and/or hemorrhaging. These microneedles may be superior to hypodermic needles traditionally used for trans-vitreal insertion at least because the microneedles afford greater precision during insertion and injection. For example, microneedles used for subretinal injection may be designed to traverse only the sclera, choroid, and RPE (and in some cases the conjunctiva) to access the subretinal space. This distance may be about 1 mm to 2 mm. However, conventional hypodermic needles used for trans-vitreal injection must be at least 2 cm to 3 cm long in order to properly traverse the vitreous humor and other tissues in the eye. This improved precision may enable more targeted injections, while also reducing risk of complications such as tissue damage and hemorrhage.

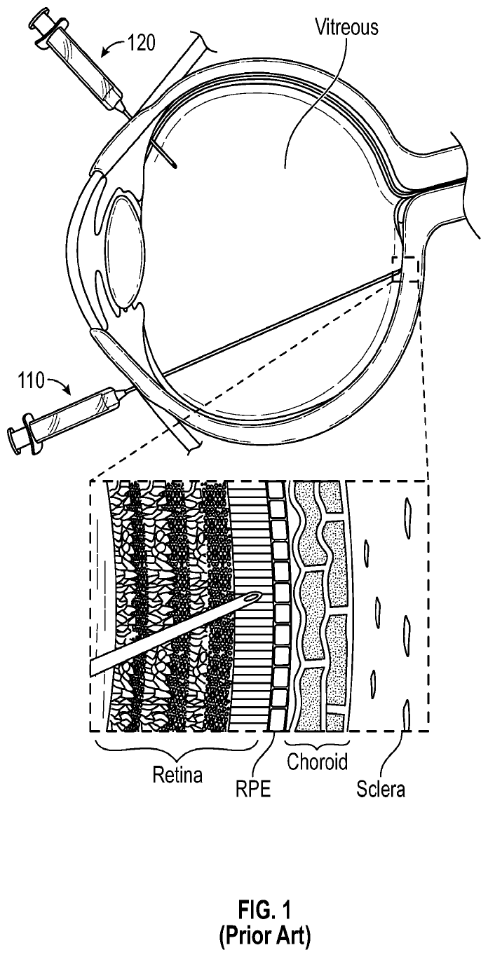

FIG. 1 illustrates a trans-vitreal injection 110, which is the most common approach for delivering therapeutic agents to the subretinal space, and an intravitreal injection 120, which is another common method for delivering therapeutic agents to the retina. Here, a standard hypodermic needle is passed through the vitreous and retina, into the subretinal space to deliver a therapeutic agent therein. As used herein, the term “vitreous” refers to the gel-like fluid that fills the eye, having fibers attaching to the retina. The term “retina” refers to the layer at the back of the eye containing photosensitive cells, which is responsible for triggering nerve impulses that pass to the brain via the optic nerve to form visual images. Unfortunately for patients, this procedure is invasive and can often result in complications.

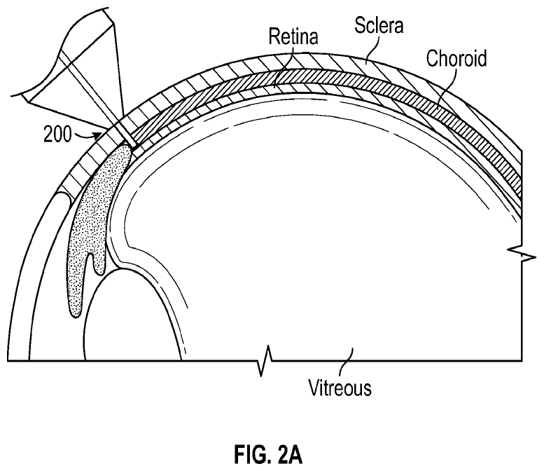

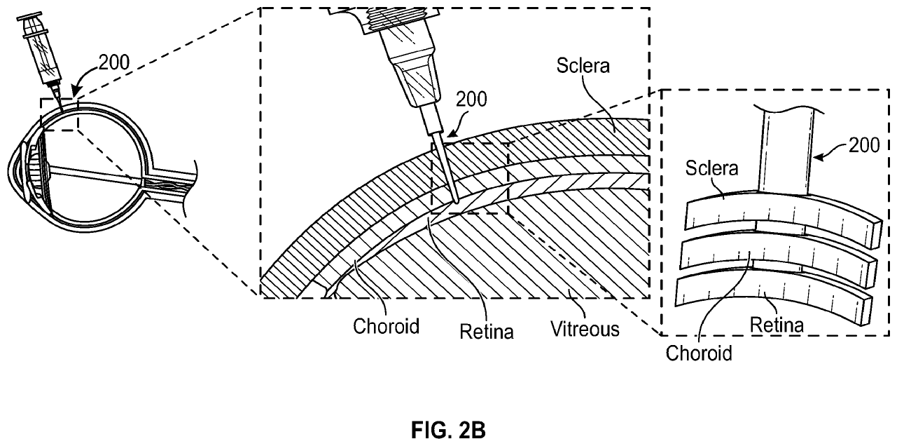

With the present disclosure, improved microneedles, apparatus, and methods are adapted for trans-scleral insertion and injection into the subretinal space. In some embodiments, this is accomplished by inserting a microneedle 200 with optimized dimensions into the eye of a patient through the conjunctiva, sclera, and choroid layers of the eye without a retinotomy, as shown in FIGS. 2A-2B. As used herein, the term “conjunctiva” refers to the mucous membrane covering the front of the eye and lining the inside of the eyelids. The term “sclera” refers to the white outer layer covering a majority of the outside of the eyeball. The terms “choroid” or “choroid layers” refer to the vascular layer of the eye between the sclera and the retina.

The safety and efficacy of the methods of trans-scleral subretinal injection disclosed herein may be dependent on the insertion site for the microneedle. In embodiments, the insertion site for the microneedle is selected to avoid puncturing large conjunctival, choroidal, and retinal blood vessels, particularly the choroidal blood vessels. For example, the eye may be imaged to identify target sites having a needle path in which no large conjunctival, choroidal, and retinal blood vessels exist. As used herein, “large” blood vessels refer to non-capillary blood vessels, or blood vessels greater than 25 μm in diameter. After an optimal target site is identified, the microneedle may be inserted at said target site.

In embodiments, the microneedle is inserted at the peripheral retina, mid-periphery, or posterior retina. As used herein, “peripheral retina” refers to the area of the retina near the limbus, “mid-periphery” refers to the area near the equator of the eye, and “posterior retina” refers to the area of the retina near or at the macula.

Following insertion of the microneedle, fluid is injected through a lumen of the microneedle into the subretinal space of the eye. To ensure that the injected fluid is properly targeting the subretinal space, the microneedle should not pass through the retina and into the vitreous. For example, the microneedle may be inserted such that the tip opening of the microneedle lumen is adjacent to the interface of the retinal pigment epithelium layer, i.e., does not penetrate deeper than the outer nuclear layer of the retina.

In some preferred embodiments, the method of insertion and injection also involves stabilizing the eye before and during the inserting of the microneedle. In some embodiments, the eye may be stabilized via a gentle suction being applied to the eye. In some embodiments, the eye may be stabilized by manual means, e.g., tweezers or forceps. In some embodiments, the method of insertion and injection involves stabilizing the microneedle within an insertion apparatus prior to injection.

### Therapeutic Agents

The present methods and insertion systems can be used to deliver essentially any suitable therapeutic agent. The “therapeutic agent” may be referred to herein as a drug, an active agent, an API, or an agent of interest. It may be a prophylactic, therapeutic, or diagnostic agent known in the art to be useful in medical or veterinary ophthalmic applications.

Non-limiting examples of therapeutic agents useful in the present methods include drugs for treating degenerative eye diseases, such as age-related macular degeneration, macular edema, diabetic retinopathy, Leber's congenital amaurosis, retinitis pigmentosa, or glaucoma. In some embodiments, the therapeutic agent is selected from suitable proteins, peptides, and fragments thereof, which can be naturally occurring, synthesized or recombinantly produced. The API may be selected from small molecules and larger biotechnology produced or purified molecules (e.g., peptides, proteins, DNA, RNA).

In some embodiments, the therapeutic agent includes stem cells, differentiated cells, viruses, phages, gene vectors, nanoparticles, microparticles, antibodies, proteins, small molecules, retinal prosthetics, or artificial retina. As used herein, the term “artificial retina” refers to implantable electronic device(s) designed to stimulate the sensation of vision, as known in the art.

### Microneedles

The microneedles useful in the present methods can be adapted from those known in the art. The microneedles may be made of essentially any suitable biocompatible material, which may be a metal, glass, polymeric, or ceramic material. In some embodiments, the microneedles are constructed as hollow tubular structures, configure for passage of a fluid through a bore, or lumen, which extends from a base end to a tip end portion of the microneedle. The microneedle may have a straight shaft and a beveled tip end portion. Other geometries are envisioned, however.

The microneedles may be operably associated with a fluid reservoir and means for injection as known in the art, e.g., a syringe. For example, the therapeutic agent may be contained within an external reservoir and delivered via a lumen of the microneedle.

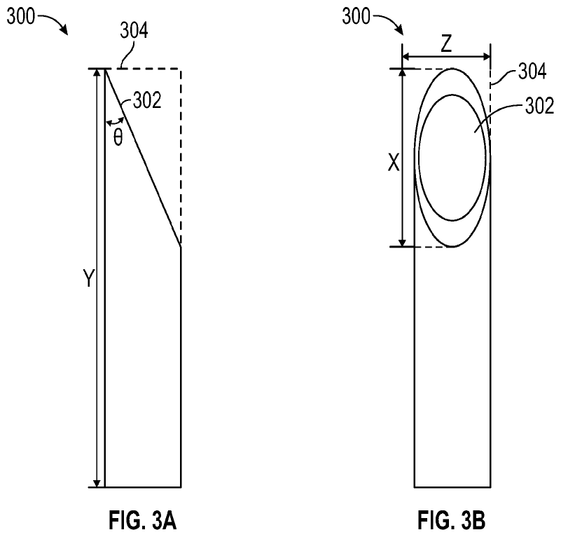

FIGS. 3A-3B illustrate one embodiment of a microneedle 300 having a lumen 302 through which the therapeutic agent may be administered. In a preferred embodiment, the microneedle 300 has a beveled tip 304, such that the length Y and width Z of the microneedle 300, the bevel angle θ of the tip 304, and the length of the bevel tip X are selected to optimize insertion and delivery of the therapeutic agent to the subretinal space. In particular, the width of the microneedle is selected to minimize bleeding at the injection site. As used herein, the “width” of the microneedle refers to the average width Z of the portion of the microneedle that is inserted into the eye. In some embodiments, the microneedle width may be between 50 μm and 1 mm, such as between 50 μm and 500 μm, 50 μm and 200 μm, 50 μm and 150 μm, 75 μm and 125 μm, or 100 μm. Other widths may be suitable, however, for use in some methods described herein. In one embodiment, the microneedle width is between 50 μm and 100 μm.

The bevel angle of the tip of the microneedle may be selected to improve penetration, thereby minimizing damage or deformation to sclera and choroid upon insertion. The beveled tip 304 may also be effective to minimize bleeding at the injection site. In various embodiments, the bevel angle may be an angle between 0° and 90°, and in particular, between 30° and 70°, 40° and 65°, 50° and 60°, or 55°. Other bevel tip angles may be suitable, however, for use in the methods described herein.

The length of the microneedle may be selected to control the penetration depth of the microneedle. As used with regard to the microneedle, the term “length” refers to the length of the microneedle protruding from the needle hub, i.e., the length of the portion of the microneedle that is inserted into the eye. In a preferred embodiment, the length of the microneedle is approximately the same as the desired penetration depth (i.e., the distance between the surface of the eye and the subretinal space), accounting for some deformation of the scleral and/or conjunctival surface. By selecting the length of the microneedle so as to control the penetration depth, full penetration of the retina may be prevented, thereby preventing complications resulting from the injection. In some embodiments, the length of the microneedle may be between 300 μm and 2 mm, such as between 500 μm and 1.5 mm, between 700 μm and 1.3 mm, between 800 μm and 1.2 mm, or about 1 mm. One skilled in the art would recognize that these lengths are suitable for use in the typical adult human eye. If an eye of a different size is used (e.g., the eye of a human child), these lengths should be scaled relative to the combined thickness of the sclera and choroid of the eye.

In embodiments, the length of the bevel tip of the microneedle may also be optimized for precise insertion and delivery of the active agent. The length of the bevel tip may be between 10 μm and 300 μm, such as between 25 μm and 250 μm, 50 μm and 200 μm, 75 μm and 150 μm, or 100 μm and 125 μm. Other tip lengths may be suitable, however, for use in the methods described herein.

### Injection Apparatus

The present methods can be carried out by any suitable means for inserting a microneedle and injecting a fluid into the subretinal space of an eye using a trans-scleral approach. In some preferred embodiments, a system, or apparatus, is provided to consistently and accurately guide the microneedle through a suitable area of the sclera and choroid and to the subretinal space and inject the fluid therein, in such a manner so as to minimize damage to the sclera, choroid, and/or retina and/or to limit or prevent excessive bleeding of the same.

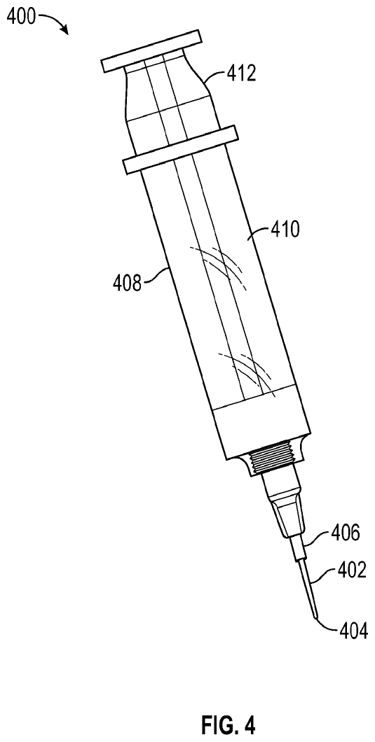

FIG. 4 illustrates one embodiment of an injection apparatus 400 for administering a therapeutic agent into the subretinal space of a patient's eye. The injection apparatus includes a microneedle 402 having a lumen 404 and extending from a needle hub 406. The injection apparatus further includes an injection portion 408 attached to the needle hub, the injection portion 408 having a fluid reservoir 410 and a means for driving the fluid 412 from the reservoir 410 into and through the lumen 404 of the microneedle, thereby injecting the fluid into the subretinal space following insertion of the microneedle. The means for driving fluid 412 may be, for example, a syringe or other similar mechanisms known in the art. In embodiments, the microneedle 402 is configured to be inserted through the conjunctiva, sclera, and choroid layers of the eye, but not into the vitreous. In other embodiments, the microneedle 402 is configured to be inserted into the eye without puncturing large conjunctival, choroidal, and/or retinal blood vessels.

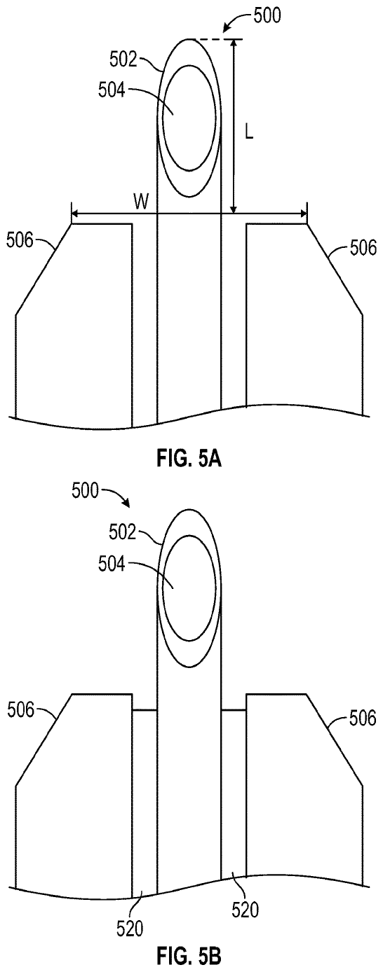

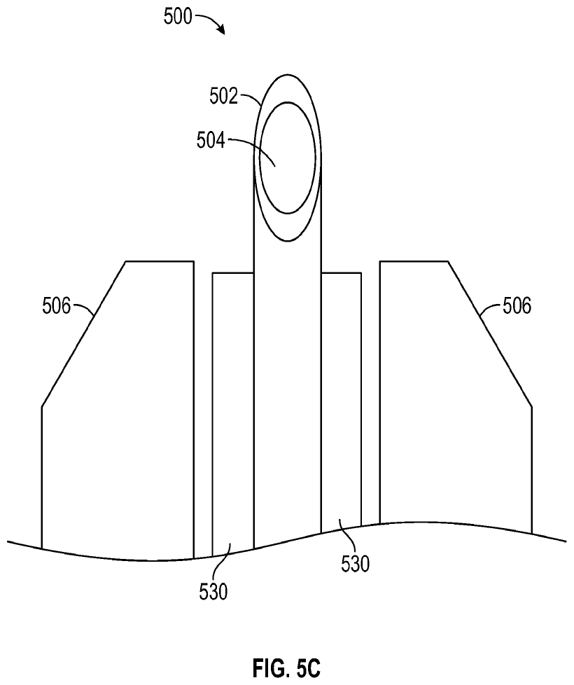

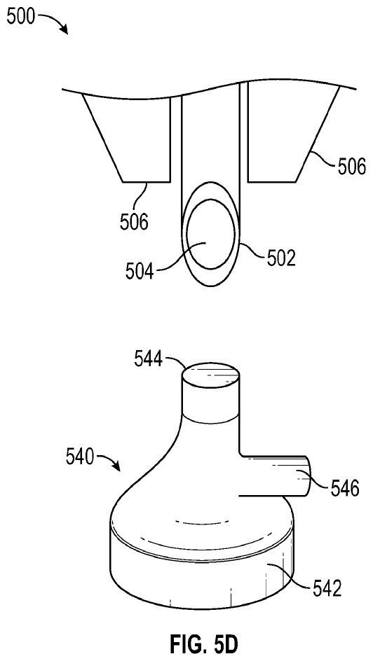

Referring now to FIGS. 5A-5B, aspects of the insertion apparatus are shown in greater detail. FIG. 5A is a cross-sectional depiction of the insertion apparatus of FIG. 4, specifically an insertion apparatus 500 having a microneedle 502 disposed within a needle hub 506. In some embodiments, the width W of the needle hub 506 at its interface with the ocular surface is minimized to reduce deformation of the ocular tissue surface. In some embodiments, the width W depends on the length L of the microneedle, such that the ratio of W/L is between 0.1 and 10, such as between 0.2 and 5, 0.5 and 3, 0.8 and 1.5, or about 1.

In embodiments, the injection apparatus 500 further includes an imaging system 520, as shown in FIG. 5B. In this preferred embodiment, the imaging system 520 is incorporated into the injection apparatus 500, such that ocular images may be obtained in real-time as the microneedle penetrates the ocular tissue. In other embodiments, the imaging system is separate from the injection apparatus 500. The imaging system 520 may be optical coherence tomography (OCT), ultrasound, or other tissue imaging techniques known in the art that are capable of ensuring there are no large conjunctival, choroidal, or retinal blood vessels in the path of the microneedle at the injection site.

In embodiments, the injection apparatus 500 further includes a microneedle stabilizer 530, as shown in FIG. 5C. Stabilization of the injection apparatus 500 and the components thereof is critical to ensure successful insertion and injection. Stabilization may be achieved by applying gentle suction to the ocular tissue surface, applying an adhesive material to the ocular tissue surface, or other methods effective to fix the position of the microneedle relative to the eye. Movement of the microneedle relative to the eye during the procedure may increase the risk that the injection does not properly target the subretinal space, and/or may increase the risk of complications to the patient, which may include damage to the patient's eye, an enlarged incision, and/or hemorrhaging. For example, a surgeon's hand tremor during injection may increase the likelihood of a choroidal hemorrhage.

In a preferred embodiment, the microneedle stabilizer 530 is disposed between the microneedle 502 and the needle hub 506 so that the microneedle 502 remains stationary during insertion. In other embodiments, the injection apparatus 500 may be mounted on a micropositioner or robotic device to perform the insertion and/or injection, as an alternative to a manual procedure. In further embodiments, alternative methods for stabilizing the microneedle and/or injection apparatus are employed, as would be understood by those skilled in the art.

In embodiments, the injection apparatus 400 further comprises an eye stabilizer 540, as shown in FIG. 5D. As described with respect to FIG. 4C, stabilization and minimization of movement during the injection process is critical. Absent stabilization, eye movement during the procedure may increase the risk of complications, such has choroidal incision and/or excessive bleeding.

In a preferred embodiment, the eye stabilizer 540 includes an eye cup 542 to be placed on the sclera to stabilize the eye. The eye stabilizer 540 further includes an injection channel 544 through which the microneedle may be inserted, such that the eye cup 542 is placed on the sclera over the injection site. Also included in the eye stabilizer 540 is a vacuum channel 546, which may be affixed to any device capable of applying gentle suction in order to stabilize the eye during the injection process.

In some other embodiments, the eye stabilizer 540 does not have an injection channel, such that the eye cup 542 is placed on the sclera opposite the injection site to stabilize the eye. In some other embodiments, alternative methods for stabilizing the eye are employed, such as using tweezers or forceps, or applying an adhesive material to the ocular tissue surface and/or to a surgical glove. Other methods of eye stabilization, as would be understood by those skilled in the art, may also be used.

### Dispersion

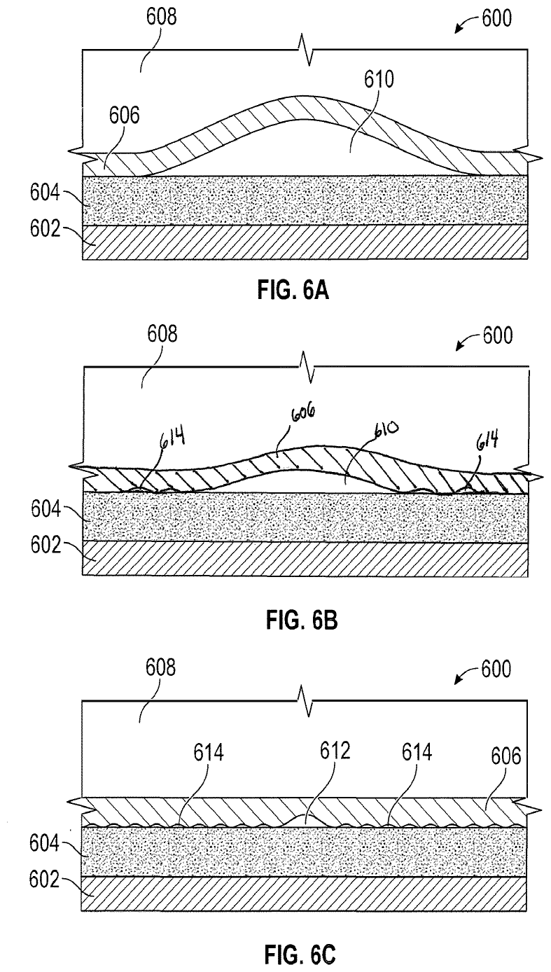

The present methods and insertion apparatus may be effective to improve the dispersion characteristics of fluid injected into the subretinal space. With conventional methods of subretinal injection, as shown in FIG. 6A, most or all of the injected fluid is retained in a subretinal bleb 610, which creates significant separation between the retinal and RPE. As used herein, the term “bleb” refers to a fluid-filled area of the subretinal space that has a separation between the retina and RPE of at least 100 μm. This is undesirable because greater separation of the retina and the retinal pigment epithelium is associated with risk of tissue damage and results in less spread of the injected fluid away from the site of injection.

The percentage (P) of fluid injected and retained in the subretinal space that is within a bleb can be calculated based on the volume of fluid injected and retained (A) in the subretinal space and the volume of the bleb(s) (B) formed in the subretinal space. This percentage (P) is equal to B/A×100%. To clarify, the fluid injected and retained in the subretinal space does not include fluid that was injected but was not retained in the subretinal space, due to, for example, leakage into the vitreous. With conventional methods of subretinal injection, P approaches 100%.

However, it has been discovered that the percentage of fluid retained in the subretinal space may be much less than 100% when injections are performed according to certain conditions. For example, fluid injected according to the methods disclosed herein may travel away from the injection side towards the posterior retina and/or macula without forming a bleb, as shown in FIG. 6B. As used herein, the term “macula” refers to the area surrounding the fovea near the center of the retina. The term “fovea” refers to the area in the middle of the retina providing the highest level of visual accuracy. In some embodiments, a collection of fluid 612 may form at the injection site, with dispersions 614 away from the injection site, such that the retina remains attached to the RPE between the collection of fluid 612 and the dispersions 614.

The collection of fluid 612 may also have a percentage (P) of fluid retained as compared to the total injection volume, as discussed with respect to FIG. 6A. In some embodiments, P is less than 90%, such as 75%, 60%, 50%, 40%, 30%, 25%, 20%, or 10%. In some embodiments, P may be between 10% and 75%, such as between 20% and 60%.

In embodiments, achieving the desired fluid dispersion pattern involves injecting the fluid at a flow rate of from 0.1 μL/s to 50 μL/s, such as from 0.3 μL/s to 20 μL/s, 0.5 μL/s to 15 μL/s, 1 μL/s to 10 μL/s, 3 μL/s to 8 μL/s, or about 5 μL/s.

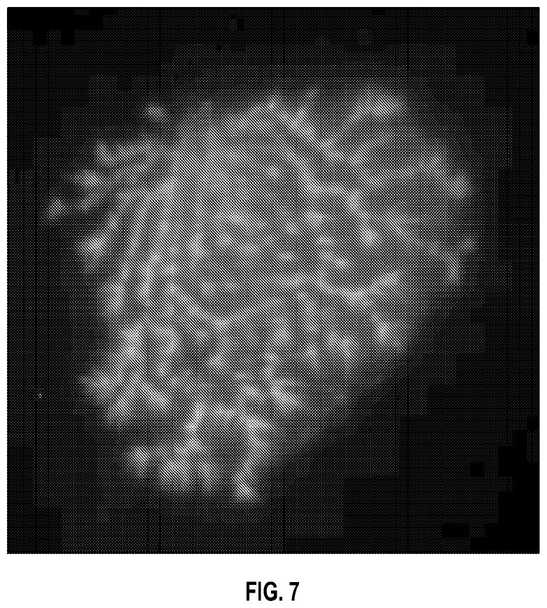

In embodiments, injecting fluid according to the methods disclosed herein is effective to produce the bifurcated dispersion pattern shown in FIG. 7. In some embodiments, the bifurcated dispersion pattern may be an intricate tree-like pattern stemming from circumferential spread of the fluid in the subretinal space. In other embodiments, the circumferential dispersion of the fluid may be less defined, but not to the extent that a subretinal bleb is formed.

The invention can be further understood with reference to the following non-limiting examples.

### Example 1: High-Precision Microneedle Injector

To develop microneedle-based methods to target injection into the subretinal space, work was performed on rodent eyes, which are especially challenging to work with due to their small size, making precise control over injection critically important. Because precision over the injection process is even more important in the subretinal space, e.g., compared to the suprachoroidal space, initial studies in the rodent eye provided a good starting point for developing the precise control over injection needed for subretinal injection.

An injector was developed that included an ultra-small hollow glass microneedle measuring 160 μm in length for rats and 260 μm for guinea pigs. A needle hub to restrict scleral deformation at the injection site was also used. A needle tip length of 110 μm and bevel angle of 55° optimized insertion without leakage. Additionally, a probe was used to secure the eye by applying gentle vacuum.

### Injector Design and Fabrication

To fabricate hollow microneedles, fire-polished aluminosilicate glass pipettes (O.D. 1 mm, Sutter Instrument, Novato, CA, USA) were pulled using a micropipette puller (P-97, Sutter Instrument). The resulting microneedles were beveled at desired angle utilizing a beveler device (BV-10, Sutter Instrument). Ethanol was then flushed through the microneedles followed by 2 flushes of DI water to clear the lumen from glass debris. Finally, microneedles were individually housed in a 12 mm-long piece of stainless-steel tubing (O.D. 1.47 mm, wall thickness 0.2 mm, McMaster-Carr, Douglasville, GA, USA) and connected to a 10 μl Hamilton syringe (#7653-01, Hamilton, Reno, NV, USA) via a fine screw fitting (M3-0.1, Base Lab Tools, Stroudsburg, PA, USA). The extremely small thread on the screw fitting enabled fine adjustment of microneedles length protruding from the tubing by moving the steel tubing forward and backward along the needle length.

The needle hub and vacuum eye stabilizer were designed via computer-aided design (Solidworks, Waltham, MA USA) and fabricated using a 3D-printer device (SLA Form 2, Formlabs, Somerville, MA, USA). Because of their contact with ocular surfaces, these parts were printed with the highest resolution to provide a smooth surface finish, which was confirmed by inspection through a stereomicroscope (Olympus SZX16, Olympus, Tokyo, Japan)

### Microneedle Injector Development

Microneedle dimensions were scaled down by fabricating microneedles out of glass using techniques adapted from micropipettes used to inject material into individual sells (e.g., those used for in vitro fertilization). While glass microneedles were easy to fabricate and handle, and did not break during use, ultra-small microneedles could be fabricated out of other materials using other methods (Kim et al. Intrastromal delivery of bevacizumab using microneedles to treat corneal neovascularization. Investigative ophthalmology & visual science. Davis et al. Hollow metal microneedles for insulin delivery to diabetic rats. IEEE Transactions on Biomedical Engineering. 2005; 52(5):909-15.). Using a micropipette puller and beveler to grind the needle tip at a pre-determined angle, microneedles with lengths as short as 100 μm with bevel angles of 30°-65° were produced.

Reliable microneedle insertion in rodents requires (1) using a needle with dimensions similar to that of rodent eyes, and (2) precise control over the depth of penetration into ocular tissue. Precise tissue penetration depth was achieved by controlling interactions at the interface between the injector device and the elastic scleral tissue. Optimization of this interaction was critical, because the margin of error in rodent eyes is extremely small, so a penetration error of just tens of microns can cause the needle tip opening to miss the SRS. Therefore, a series of studies were performed to optimize factors that determine penetration accuracy, including (1) microneedle length, tip sharpness, and tip length, (2) needle hub design, and (3) eye stabilization.

### Microneedle Tip Sharpness

The microneedle bevel angle was varied between 30° to 65°, and 55° was determined to be the optimal bevel angle. Blunter tips (e.g., 65°) were too dull and did not penetrate sclera completely in this animal model. Sharper tips (e.g., 30° and 45°) enabled easier insertion but caused fluid leakage while injecting because their tip opening spanned the scleral tissue thickness in this animal model.

### Microneedle Tip Length

The microneedle tip length was also varied between 90 to 150 μm. Independent of the microneedle tip length, all microneedles with a 55° bevel angle penetrated into the scleral tissue well. However, microneedles with longer tip lengths (e.g., 130 and 150 μm) exhibited extraocular leakage, where the microneedles with shorter tip lengths (e.g., 90 and 110 μm) reliably injected fluid. Because the 90 and 110 μm microneedles performed equally well in this animal model, the 110 μm microneedle was selected over the 90 μm microneedle because it was less fragile.

### Needle Hub Design

It was also hypothesized that poor penetration of the microneedles may arise from unwanted interactions between the injection device and the sclera at the tissue interface. The high aspect ratio needle hub width (β) to the needle length (α) was suspected to be giving rise to the poorly controlled microneedle-tissue interaction that was impeding microneedle penetration.

Two design parameters based on the hub width-to-needle length ratio

$$( \frac{β}{α} )$$

were compared:

$$( \frac{β}{α} >> 1 ) , \qquad ( 1 )$$

$$and$$

$$( \frac{β}{α} \sim 1 ) . \qquad ( 2 )$$

The former design had variable success due to the resulting tissue deformation across large areas not centered around the site of insertion, exposing sclera to a partial needle length. The large hub's footprint also made it difficult to determine the insertion angle at the injection site. In contrast, the

$$\frac{β}{α} ≅ 1$$

hub shape resulted in limited tissue deformation and improved visualization to facilitate perpendicular microneedle insertion. However, even the optimal

$$\frac{β}{α} ≅ 1$$

hub shape only resulted in microneedle puncture into the sclera of 50% of eyes and successful injection in 28% of eyes.

### Eye Stabilization

Eye movement was another confounding factor that could disrupt MN penetration dynamics. Given the sub-millimeter length scales of the injection process, even the slightest eye movement could cause misalignment at the microneedle-tissue, thereby reducing the effectiveness of optimizing microneedle and hub design. It was therefore hypothesized that stabilizing the eye to prevent movement during injection would improve success rate. As such, a probe that applied a gentle vacuum to the cornea was designed, which secured the eye in place during injection.

### Example 2: A Non-Surgical Method for Subretinal Delivery by Trans-Scleral Microneedle Injection

A microneedle was inserted into the eye and delivered material into the subretinal space (SRS) by penetrating across sclera and choroid without requiring vitrectomy or retinotomy. It reliably administered into the SRS with no incidence of retinal perforation and little or no choroidal bleeding without retinal toxicity. Tissue damage was microscopically localized to the microneedle penetration site in the retinal periphery. Trans-scleral microneedle injection may therefore provide a safe non-surgical method of SRS injection, as compared to conventional therapies.

### SRS Injector Design and Fabrication

To fabricate hollow microneedles, fire-polished aluminosilicate glass pipettes (O.D. 1 mm, Sutter Instrument, Novato, CA, USA) were pulled using a micropipette puller (P-97, Sutter Instrument). The resulting microneedles had a tip bevel angle of 55° and a tip length of 110 μm, achieved utilizing a beveler device (BV-10, Sutter Instrument). Ethanol was then flushed through the microneedles followed by 2 flushes of DI water to clear the lumen from glass debris. Finally, microneedles were individually housed in a 12 mm-long piece of stainless-steel tubing (O.D. 1.47 mm, wall thickness 0.2 mm, McMaster-Carr, Douglasville, GA, USA) and connected to a 10 μl Hamilton syringe (#7653-01, Hamilton, Reno, NV, USA) via a fine screw fitting (M3-0.1, Base Lab Tools, Stroudsburg, PA, USA). The extremely small thread on the screw fitting enabled fine adjustment of microneedle length protruding from the tubing by moving the steel tubing forward and backward along the needle length.

The needle hub and vacuum eye stabilizer were designed via computer-aided design (Solidworks, Waltham, MA USA) and fabricated using a 3D-printer device (SLA Form 2, Formlabs, Somerville, MA, USA). Because of their contact with ocular surfaces, these parts were printed with the highest resolution to provide a smooth surface finish, which was confirmed by inspection through a stereomicroscope (Olympus SZX16, Olympus, Tokyo, Japan).

An injection method for trans-scleral SRS delivery in a safe and reliable manner was assessed. The approach relied on two key outcomes: (1) precise placement of the needle tip in the SRS, and (2) minimized disruption of ocular tissue. To achieve these desired outcomes, the following features were incorporated into the injection technique: (1) a microneedle with a tightly controlled length to match the thickness of the sclera and choroid, (2) a vacuum probe to stabilize the eye, and (3) perpendicular microneedle insertion into the eye. These elements cumulatively achieved precise needle placement because the controlled microneedle length enabled penetration across the sclera and choroid into the SRS, but physically inhibited deeper penetration into the neural retina.

### Trans-Scleral SRS Injection

To expose the scleral surface, the eye was proposed using the latex glove method. A custom-made 3D printed eye probe was then placed on the inferior cornea-sclera through which a gentle vacuum (Vacuum pump AIRPO D2028B, Karlsson Robotics, Tequesta, FL USA) was applied to secure the eye during injection. The microneedle was perpendicularly inserted into the eye 1-2 mm posterior to the limbus on the superior side and liquid formulations were injected slowly (˜0.3 μl/s) by pushing the plunger. The microneedle was kept in place for 30 s after injection to prevent reflux, after which the microneedle and latex glove were removed, and the vacuum was turned off.

Prior to injection, the microneedle length was adjusted to 120 μm for mice, 220 μm for rats and 300 μm for guinea pig injections. Each injection took about 1 min, starting from the glove proptosis until the vacuum was turned off and the glove removed. To assess injection success, fundus and OCT images were collected immediately (within 1 min) after injection.

### Ocular Bleeding

Development of trans-scleral injection into the SRS for clinical application has been limited by the expectation that choroidal puncture bears a high risk of uncontrolled hemorrhage.

Hypodermic Needles Vs. Microneedles

The extent of choroidal bleeding was found to be proportional to the degree of damage to the choroidal vasculature, which is also proportional to needle size. Hemorrhage therefore could be reduced or eliminated by using a small enough needle to minimize rupture of choroidal vessels.

Incidences of ocular bleeding were assessed following insertion of needles of various sizes across the sclera and choroid of a rat eye in vivo. Needles were repeatedly inserted up to 4 times per eye, and it was observed that all eyes into which hypodermic needles with an outer diameter of 210 μm (33 G), 310 μm (30 G) or 410 μm (27 G) were inserted experienced intraocular and/or extraocular hemorrhage after just one or two insertions. In contrast, among eyes that received insertion of a microneedle with outer diameter of 110 μm, there were no eyes exhibiting intraocular or extraocular hemorrhage, even after four insertions per eye. This finding shows that the small size of the MN avoided ocular hemorrhage even after repeated MN insertions, while hypodermic needles led to hemorrhage in all cases.

In addition to the cumulative effect of multiple insertions, it was also observed that there was a 67% (6/9), 60% (6/10), or 75% (6/8) chance of ocular hemorrhage after each insertion with a 33 G, 30 G, or 27 G needle, respectively. Considering extraocular versus intraocular hemorrhage, hypodermic needles led to intraocular hemorrhage in 22% to 62% of eyes and extraocular hemorrhage in 30% to 75% of eyes. In contrast, intraocular or extraocular hemorrhage was not observed in eye punctures with microneedles.

### Characterization of Ocular Bleeding

During microneedle device development, ocular bleeding was observed due to needle insertion. To further assess this bleeding during SRS injection, bleeding was measured in 31 rat eyes that had received trans-scleral SRS injections. Serial OCT images taken immediately after injection were used to calculate bleeding by multiplying the thickness of each image (120 μm) by the sum of cross-sectional areas of subretinal bleeding measured from individual OCT image. Bleeding was easily identifiable as an opaque region within the subretinal bleb. Blebs without bleeding appeared clear (black). All measurements were made using ImageJ (NIH, USA).

Contrary to the general paradigm that any penetration involving choroidal vascular may cause severe hemorrhage (Gerding, A new approach towards a minimal invasive retinal implant. Journal of neural engineering. 2007; 4(1):S30), it was observed that choroidal hemorrhage after SRS microneedle injection was rare, and when it did occur it was minimal, self-contained, and localized. This was likely achieved by the ultra-small size of microneedles that reached the SRS by making a microscopic rupture through choroid vasculature, thereby minimizing the incidence and extent of bleeding. It is also plausible that perpendicular insertion and eye stabilization contributed to low bleeding incidence, albeit effects of insertion angle and eye movement were not separately tested.

Although the bleeding was minimal in all cases, it was still noteworthy that 45% of injections caused no detectable bleeding while other injections did, despite having been performed by identical microneedles. This may have been attributable to the size of the ruptured vasculature. The choroid consists of an intertwined network of various size blood vessels (Ferrara et al., Investigating the choriocapillaris and choroidal vasculature with new optical coherence tomography technologies. Progress in retinal and eye research. 2016; 52:130-55), and perforating the choroid at a region mostly consisted of small choriocapillaris would likely cause considerably smaller bleeding compared to when a large vessel is nicked. This finding offers a strategy to reduce such risk in humans even further by identifying an optimal puncture site ideally empty of medium and large blood vessels, prior to the injection, using noninvasive imaging instruments such as OCT or ultrasound.

### Subretinal Delivery by Trans-Scleral Microneedle Injection

SRS injection involves crossing the choroid without hemorrhage as well as precisely placing the needle tip within the SRS without deeper penetration into neural retina. Having reduced needle width to avoid hemorrhage, needle penetration depth was subsequently controlled by (1) optimizing microneedle length to only penetrate the sclera and choroid, and (2) minimizing factors that could disrupt microneedle placement in the tissue.

Microneedle length was optimized by considering the anatomy of our three animal models—mouse, rat, and guinea pig—by accounting for the total thickness of conjunctiva, sclera, and choroid that the microneedle must cross to reach the SRS. This yielded optimal MN lengths of 120 μm, 220 μm and 300 μm for the mouse, rat and guinea pig, respectively, which was precisely controlled by mounting the MN in an adjustable holder with a fine adjustment screw to control the microneedle exposed length. All microneedles used also had a tip length of 110 μm and a tip bevel angle of 55°. Movement after precise microneedle placement in the tissue during microneedle insertion and injection was also minimized by (1) use of a tapered needle hub to facilitate perpendicular insertion, and (2) eye stabilization.

The optimized injection technique for SRS delivery in mice, rats, and guinea pigs were used to assess successful SRS delivery based on formation of a fluid bleb in the SRS. 65 SRS injections in rats achieved bleb formation 86% of the time, indicating general reliability of the SRS injection technique. Bleb formation was readily evident by brightfield fundus imaging, and optical coherence tomography (OCT) imaging further confirmed bleb formation localized in the SRS. Similar studies were carried out in mice and guinea pigs, which yielded similar findings. Consistent with SRS injection by other methods, the induced subretinal bleb was transient and self-resolved within 24 h post-injection.

### Acute and Long-Term Safety Examinations

Aiming to better understand procedure's safety beyond choroidal bleeding, a broader analysis post-mortem at several timepoints was conducted to identify acute and long-term outcomes. Histological examinations revealed nothing notable over the course of six weeks anywhere in the bleb region other than at the site of microneedle puncture. At the puncture site, there was evidence of penetration across choroid and RPE, but no puncture across retina was seen and this microscopic puncture did not expand over time. A mild macrophage reaction was also observed at the puncture site 24 h post-injection, but disappeared within 10 days. There was no evidence of neovascularization or apoptosis. In rare cases, injection into inner retinal layers was seen near injection site that was associated with retinal damage.

### Example 3: Bifurcation Pattern Formation in the Subretinal Space of the Eye

Injection into the SRS enables targeted gene therapy and drug delivery to diseased retinal cells to treat a variety of vision-threatening eye disorders. This approach, however, is constrained by the formation of a subretinal blister (i.e., bleb) due to retinal detachment from the underlying retinal pigment epithelium, which is generally seen as an uncontrolled and inevitable outcome (Ding et al., AAV8-vectored suprachoroidal gene transfer produces widespread ocular transgene expression. The Journal of clinical investigation. 2019; 129(11):4901-11; Baldassarre et al., Subretinal delivery of cells via the suprachoroidal space: Janssen trial. Cellular Therapies for Retinal Disease: Springer; 2017. p. 95-104). This blister generates safety concerns due to strain and detachment of the fragile retinal structure (Ochakovski et al., Retinal gene therapy: surgical vector delivery in the translation to clinical trials. Frontiers in neuroscience. 2017; 11:174) and efficacy limitations due to insufficient injection spread in the SRS (Yiu et al., Suprachoroidal and subretinal injections of AAV using transscleral microneedles for retinal gene delivery in nonhuman primates. Molecular Therapy-Methods & Clinical Development. 2020; 16:179-91). However, when performing SRS injection according to the methods otherwise disclosed herein, formation of fluid propagation in the SRS involving bifurcation pattern was observed.

Injection of particle suspensions into the SRS according to these methods lead to formation of stringy fingers of fluid flow along the subretinal tissue interface that repeatedly bifurcate as they grow. This fingering flow initially led to micro-detachment of ocular layers that ultimately expanded into full delamination of the retina to form blisters. It was also observed that increased injection flow rate could increase subretinal spread of injected fluid while inhibiting blister growth, thereby addressing both the safety and efficacy concerns associated with current SRS injections. These observations introduce a new class of flow instability problems where multiple physics of fluid, viscoelastic and adhesion mechanics intersect in the presence of biological complexity. These findings also have significant implications for safer and more efficacious ophthalmic treatments enabled by SRS injection that maximizes injection spread while minimizing blister formation.

### Intricate Bifurcating Patterns Emerge Following Injection of Solid Suspensions

The spread behavior of fluid in the SRS of rodent eyes in vivo was characterized by employing advanced ocular imaging techniques. Fluorescent agents were added to the injection media to provide better visualization and more detailed flow tracking. Injection of a solution containing fluorescein into the SRS ubiquitously resulted in delamination of retina from underlying tissue and formation of a fluid blister as seen in fundus and OCT images.

Motivated by (1) the unique behavior of solid-particle suspensions in interfacial flow, and (2) its relevance for gene and stem cell therapies, a dilute aqueous solution containing fluorescent nanoparticles was injected into the SRS of rat eyes. Unlike solution, suspension injection resulted in a striking observation of self-assembled pattern formation with stringy fingers that stem from the injection site and bifurcate as they grew radially. These highly branched structures emerged in all injections involving fluorescent suspensions without exception and were similarly reproduceable in a different animal model (e.g., guinea pigs).

### Pattern Fingers Form at the Subretinal Interface and May Precede Blister Formation

Histology examination of tissue sections revealed formation of crevices at the base of the retina. Crevices appeared in the regions where a blister had completely delaminated the retina as well as at the leading edge of the subretinal spread where the retina was not regionally delaminated by the blister. While the injected particles could not be seen due to dissolution during histology tissue preparation, the presence of crevices can be attributed to the traces that pattern fingers had left because those crevices were not seen in the eyes that received solution injection.

The apparent discrepancy between the patterning behavior of suspension and solution can be explained by a physical steric entrapment argument. In suspension injection, a fluid finger deforms the soft retina at the retina-RPE interface and creates a crevice. As fluid continues to flow in the newly formed finger channel, particles are sheared between the confining boundaries of the crevice and aggregate to form flocs. The particle flocs then continue to remain attached to the crevice even after blister fully delaminates the retina which is consistent with previous observation in OCT images where fingers could be seen attached to the retina in the blister region. The resulting flocs formation enables visualization of fingers by creating higher contrast in the finger channel compared to the surrounding bulk fluid. In solution injection, on the other hand, these flocs do not form when flowing soluble molecules through finger channels, and the newly formed fingers are rapidly smoothed out by the viscoelastic relaxation of the retina, thus preventing direct visualization. This mechanism is similar to the effect of dilute proppants (i.e., small solid particles) in hydraulic fracturing of shell rocks to keep the cracks open for continuous flow of oil out of the reservoir. It is therefore believed that the presence of solid particles during SRS injection facilitated imaging of the bifurcating flow patterns, but were not required to generate the bifurcating flow patterns.

### Increased Injection Flowrate Increased Patterned Flow and Suppressed Blister Growth

Equipped with this new understanding, it was explored whether fluid-solid (i.e., tissue) interactions can be manipulated to favor flow by interfacial pattern formation. It was believed that fingering flow can be increased by injecting fluids at a faster flowrate. Fluid flow in the SRS can be approximated as flow in a narrow gap given its typical aspect ratio (radius of spread/blister height r/h>>1). As such, the radial pressure gradient in the SRS follows Darcy's law, Q=−[2πrh\hat{ }3/(12μ)]∂p, in which fluid pressure is proportional to the imposed flowrate, p˜12μQ/(2πh\hat{ }3). Here, p is fluid pressure, Q indicates flowrate, r is radius of spread, h denotes blister height and μ stands for fluid viscosity. Therefore, injecting at a faster flowrate increases the pressure inside the fluid domain. This increased pressure can either accommodate the flow by deflecting the retina leading to higher blister growth, or it can expand the SRS laterally resulting in more patterned flow.

Using this elevated flowrate, patterned and blister flow coexisted at all volumes, consistent with previous observations using a slower flowrate. However, fast injection significantly increased the radial spread while suppressing blister height. Flow characteristics were measured to quantify this effect, including spread area, normal radius of spread and blister height. The increased flowrate drastically altered fluid biomechanics to accommodate flow by increased expansion of interfacial area rather than blistering deflection, despite the natural tendency of the soft retina to delaminate. In particular, increasing flowrate 20-fold increased area and nominal radius of spread by 133%±79.5% and 50.6%±27.8%, respectively, while suppressing blister height by 42.4%±17.6%, on average.

From a clinical standpoint, SRS injection of particles are of interest, but applications focus especially on adeno-associated viral (AAV) particles due to their extensive use in retinal gene therapy. Therefore, AAV vectors measuring ˜25 nm in diameter (Dalkara et al., Inner limiting membrane barriers to AAV-mediated retinal transduction from the vitreous. Molecular Therapy. 2009; 17(12):2096-102) were injected and a similar spread behavior was observed. This implies that the previously described findings with regard to suspension injection are not limited to polymeric particles, but also apply to virus delivery applications.

The flow characteristics of slow injections revealed another important fluid-solid mechanical behavior: at a certain critical volume (V≈2 μl), spread in the SRS plateaued to a critical area (A≈10 mm²) and a critical radius (R≈2 mm), beyond which the fluid was no longer capable of propagating along the subretinal interface. While the quantitative values reported here are specific to studies in the rat eye, the qualitative phenomena observed are believed to be applicable to humans and other animals. At a critical radius, the fluid pressure is balanced out by the sum of all those forces at which point flowrate approaches zero because, according to Darcy's law, fluid pressure and flowrate are directly proportional. At that point, further fluid injection can only lift up the retina.

Some embodiments of the present disclosure can be described in view of one or more of the following:

Embodiment 1. A method of administering a therapeutic agent to an eye of a patient, the method comprising: inserting a microneedle into the eye of the patient, wherein the microneedle extends through the sclera and choroid layers, but not into the vitreous, and without a vitrectomy or a retinotomy; and injecting a fluid comprising a therapeutic agent through a lumen of the microneedle and into a subretinal space (SRS) of the eye, wherein the microneedle has (i) a beveled tip with a bevel angle from 40° to 70° and (ii) an outer diameter that is less than 150 μm.

Embodiment 2. The method of Embodiment 1, wherein the bevel angle is from 50° to 60° and the outer diameter is from 75 μm to 125 μm.

Embodiment 3. The method of Embodiment 1 or 2, wherein the microneedle is inserted such that a tip opening of the microneedle is located at the interface of the retina and retinal pigment epithelium (RPE) layers without penetrating deeper than the outer nuclear layer of the retina.

Embodiment 4. The method of any one of Embodiments 1 to 3, further comprising, before inserting: imaging tissue of the eye to identify one or more target sites which have a needle path in which no large conjunctival, choroidal and retinal blood vessels exist; and selecting one of the one or more targets sites as the site for inserting the microneedle.

Embodiment 5. The method of any one of Embodiments 1 to 4, further comprising stabilizing the eye while inserting the microneedle.

Embodiment 6. The method of any one of Embodiments 1 to 5, wherein the site of the insertion is selected from the peripheral retina, mid-periphery, or posterior retina.

Embodiment 7. The method of any one of Embodiments 1 to 6, wherein the injecting is done in manner that suppresses subretinal bleb growth and guides the fluid away from the site of the insertion, and optionally toward the posterior retina and/or macula.

Embodiment 8. The method of Embodiment 7, wherein 25% to 75%, such as 50%, of the injected fluid forms a subretinal bleb.

Embodiment 9. The method of Embodiment 7 or 8, wherein the injecting produces formation of intricate tree-like bifurcating patterns that spread circumferentially from the site of the injection.

Embodiment 10. The method of any one of Embodiments 7 to 9, wherein the fluid is injected at a flowrate from 0.5 μL/s to 20 μL/s, such as from 2 to 10 μL/s.

Embodiment 11. The method of any one of Embodiments 1 to 10, wherein the therapeutic agent is effective in the treatment of age-related macular degeneration, macular edema, diabetic retinopathy, Leber's congenital amaurosis, retinitis pigmentosa, or glaucoma.

Embodiment 12. The method of any one of Embodiments 1 to 11, wherein the therapeutic agent comprises stem cells, differentiated cells, viruses, phages, gene vectors, nanoparticles, microparticles, antibodies, proteins, small molecules, retinal prosthetics, and/or artificial retina.

Embodiment 13. The method of any one of Embodiments 1 to 12, wherein an inserted portion of the microneedle has a length of between about 500 μm and 1.5 mm.

Embodiment 14. The method of claim 1, further comprising, before the inserting, selecting a length of the microneedle such that the beveled tip of the microneedle is configured to be positioned within the SRS upon complete insertion of the microneedle.

Embodiment 15. An injection apparatus for administering a therapeutic agent into a subretinal space (SRS) of an eye of a patient, the apparatus including: a microneedle which extends from a needle hub and is configured to be inserted through the sclera and choroid layers, but not into the vitreous; and an injector configured to inject a fluid comprising a therapeutic agent through a lumen of the microneedle and into the SRS following insertion of the microneedle; wherein the microneedle has (i) a beveled tip with a bevel angle from 40° to 70° and (ii) an outer diameter that is less than 150 μm.

Embodiment 16. The injection apparatus of Embodiment 15, wherein the microneedle has an outer diameter between 50 μm and 120 μm and the bevel angle is from 50° to 60°.

Embodiment 17. The injection apparatus of Embodiment 15 or 16, wherein the microneedle has a length between 500 μm and 2 mm, such as between 500 μm and 1.5 mm, between 800 μm and 1.2 mm, or about 1 mm.

Embodiment 18. The injection apparatus of any one of Embodiments 15 to 17, wherein the needle hub as a width (W) and the microneedle as a length (L), the ratio of W:L is between 0.1 and 10, such as between 0.2 and 5, between 0.5 and 3, between 0.7 and 2, between 0.8 and 1.5, or about 1.

Embodiment 19. The injection apparatus of any one of Embodiments 15 to 18, wherein the injector comprises: a reservoir for the fluid; and means for driving the fluid from the reservoir into and through the microneedle.

Embodiment 20. The injection apparatus of any one of Embodiments 15 to 19, wherein the injection apparatus further comprises: means for stabilizing a position of the microneedle relative to a target site in an eye of a patient; and/or an imaging system configured to image a target site for a microneedle insertion path in which no large conjunctival, choroidal and retinal blood vessels exist.

That which is claimed is:

1. A method of administering a therapeutic agent to an eye of a patient, the method comprising:
inserting a microneedle into the eye of the patient, wherein the microneedle extends through the sclera and choroid layers, but not into the vitreous, and without a vitrectomy or a retinotomy; and
injecting a fluid comprising a therapeutic agent through a lumen of the microneedle and into a subretinal space (SRS) of the eye,
wherein the microneedle has (i) a beveled tip with a bevel angle from 40° to 70° and (ii) an outer diameter that is less than 150 μm.

2. The method of claim 1, wherein the bevel angle is from 50° to 60° and the outer diameter is from 75 μm to 125 μm.

3. The method of claim 1, wherein the microneedle is inserted such that a tip opening of the microneedle is located at the interface of the retina and retinal pigment epithelium (RPE) layers without penetrating deeper than the outer nuclear layer of the retina.

4. The method of claim 1, further comprising, before inserting:
imaging tissue of the eye to identify one or more target sites which have a needle path in which no large conjunctival, choroidal and retinal blood vessels exist; and
selecting one of the one or more targets sites as the site for inserting the microneedle.

5. The method of claim 1, further comprising stabilizing the eye while inserting the microneedle.

6. The method of claim 1, wherein the site of the insertion is selected from the peripheral retina, mid-periphery, or posterior retina.

7. The method of claim 1, wherein the injecting is done in manner that suppresses subretinal bleb growth and guides the fluid away from the site of the insertion, and optionally toward the posterior retina and/or macula.

8. The method of claim 7, wherein from 25% to 75% of the injected fluid forms a subretinal bleb.

9. The method of claim 7, wherein the injecting produces formation of intricate tree-like bifurcating patterns that spread circumferentially from the site of the injection.

10. The method of claim 7, wherein the fluid is injected at a flowrate from 0.5 μL/s to 20 μL/s, such as from 2 μL/s to 10 μL/s.

11. The method of claim 1, wherein the therapeutic agent is effective in the treatment of age-related macular degeneration, macular edema, diabetic retinopathy, Leber's congenital amaurosis, retinitis pigmentosa, or glaucoma.

12. The method of claim 1, wherein the therapeutic agent comprises stem cells, differentiated cells, viruses, phages, gene vectors, nanoparticles, microparticles, antibodies, proteins, small molecules, retinal prosthetics, and/or artificial retina.

13. The method of claim 1, wherein an inserted portion of the microneedle has a length of between about 500 μm and 1.5 mm.

14. The method of claim 1, further comprising, before the inserting, selecting a length of the microneedle such that the beveled tip of the microneedle is configured to be positioned within the SRS upon complete insertion of the microneedle.

15. An injection apparatus for administering a therapeutic agent into a subretinal space (SRS) of an eye of a patient, the apparatus comprising:
a microneedle which extends from a needle hub and is configured to be inserted through the sclera and choroid layers, but not into the vitreous; and
an injector configured to inject a fluid comprising a therapeutic agent through a lumen of the microneedle and into the SRS following insertion of the microneedle;
wherein the microneedle has (i) a beveled tip with a bevel angle from 40° to 70° and (ii) an outer diameter that is less than 150 μm.

16. The injection apparatus of claim 15, wherein the microneedle has an outer diameter between 50 μm and 120 μm and the bevel angle is from 50° to 60°.

17. The injection apparatus of claim 15, wherein the microneedle has a length between 500 μm and 2 mm, such as between 500 μm and 1.5 mm, between 800 μm and 1.2 mm, or about 1 mm.

18. The injection apparatus of claim 15, wherein the needle hub has a width (W), the microneedle as a length (L), and the ratio of W:L is between 0.1 and 10, such as between 0.2 and 5, between 0.5 and 3, between 0.7 and 2, between 0.8 and 1.5, or about 1.

19. The injection apparatus of claim 15, wherein the injector comprises:
a reservoir for the fluid; and
means for driving the fluid from the reservoir into and through the microneedle.

20. The injection apparatus of claim 15, wherein the injection apparatus further comprises:
means for stabilizing a position of the microneedle relative to a target site in an eye of a patient; and/or
an imaging system configured to image a target site for a microneedle insertion path in which no large conjunctival, choroidal and retinal blood vessels exist.
